(19) United States
(12) Patent Application Publication
(10) Pub. No.: US 2025/0143876 A1
(43) Pub. Date: May 8, 2025

(54) DEVICES AND SYSTEMS FOR DOCKING A PROSTHETIC VALVE

(71) Applicant: EDWARDS LIFESCIENCES CORPORATION, Irvine, CA (US)

(72) Inventors: Eran Grosu, Kfar-Saba (IL); Eran Goldberg, Nesher (IL); Sean Timothy O'keeffe, Tustin, CA (US); Douglas Thomas Dominick, Irvine, CA (US); Michal Lipperman, Kfar-Saba (IL)

(21) Appl. No.: 19/016,497

(22) Filed: Jan. 10, 2025

Related U.S. Application Data
(63) Continuation of application No. PCT/US2023/027480, filed on Jul. 12, 2023.
(60) Provisional application No. 63/368,824, filed on Jul. 19, 2022.

(51) Int. Cl. A61F 2/24

(52) U.S. Cl. CPC A61F 2/2418; A61F 2/2427; A61F 2220/0075; A61F 2230/0004; A61F 2250/0098

(57) ABSTRACT

In some examples, a medical implant includes a frame and a radiopaque marker. The radiopaque marker is coupled to the strut with a recessed surface portion of the radiopaque marker positioned in opposing relation to a first concave surface of a strut portion of the frame. In some examples, at least one strut of the frame has a bore, an eyelet, and a strut portion between the bore and the eyelet. The radiopaque marker is disposed within the bore and has an edge surface with a recessed surface portion positioned in opposing relation to a first surface of the strut portion. In some examples, a medical assembly includes a delivery apparatus, a frame, and a radiopaque marker.

### CROSS REFERENCE TO RELATED APPLICATION

This application is a continuation of PCT Patent Application No. PCT/US2023/027480 filed Jul. 12, 2023, which claims the benefit of U.S. Provisional Patent Application No. 63/368,824, filed Jul. 19, 2022, each of these applications being incorporated by reference herein in its entirety.

### FIELD

The present disclosure relates to prosthetic valves and, in particular, docking stations, delivery systems, and methods for use in implanting a prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

In one specific example, a prosthetic valve can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

In some cases, it may not be possible to secure the prosthetic valve to the native annulus, for example, if the native annulus is too large or if the geometry of the native valve is too complex to allow secure implantation of the valve. One approach in these cases is to first deploy a docking station at the implantation site and then install the prosthetic valve in the docking station. The docking station can be selected to provide the necessary interface to anchor the prosthetic valve within the native annulus. Desirably, the docking station can be delivered to the implantation site with a minimally invasive procedure, which would allow the docking station to be deployed within the same procedure to deliver the prosthetic valve.

### SUMMARY

Medical implants can include radiopaque markers that enable detection of the medical implant within a patient's body using x-ray/fluoroscopy imaging. Disclosed herein are non-circular radiopaque markers that can be mounted in circular or non-circular bores formed in struts of a frame of a medical implant, such as a docking station. The non-circular radiopaque markers can engage the bores in a manner to reduce strain concentration on portions of the struts adjacent to the bores due to outward radial pressure exerted on the walls of the bores by the radiopaque markers.

A medical implant can comprise a frame and a radiopaque marker. In addition to these components, a medical implant can further comprise one or more of the components disclosed herein.

In some examples, the radiopaque marker can comprise an edge surface with a recessed surface portion and a non-recessed surface portion.

In some examples, the frame can comprise at least one strut having a strut portion with a first concave surface and a second concave surface on opposite sides of the strut portion.

In some examples, the radiopaque marker can be coupled to the at least one strut with a recessed surface portion of the radiopaque marker positioned in opposing relation to the first concave surface.

In some examples, the radiopaque marker can comprise opposite planar major surfaces, and the edge surface can extend between the opposite major surfaces.

In some examples, the radiopaque marker can be asymmetric about a plane transverse to the opposite major surfaces.

In some examples, the medical implant can comprise a docking station configured to receive a prosthetic valve, and the plurality of struts can define a valve seat for engaging the prosthetic valve when the prosthetic valve is inserted into the docking station.

In some examples, the prosthetic valve can comprise a frame and a valvular structure coupled to the frame.

In some examples, the frame of the medical implant can comprise a plurality of struts defining a plurality of cells.

In some examples, at least one strut of the plurality of struts can comprise a bore and an eyelet formed therein.

In some examples, the at least one strut can comprise a strut portion between the bore and the eyelet. A portion of a wall of the bore can form a first surface of the strut portion and a portion of the wall of the eyelet can form a second surface of the strut portion. The first and second surfaces can be opposite sides of the strut portion.

In some examples, the radiopaque marker can be disposed within the bore.

In some examples, the radiopaque marker can comprise an edge surface with a recessed surface portion and a non-recessed surface portion, and the recessed surface portion can be positioned in opposing relation to the first surface of the strut portion.

In some examples, the recessed surface portion can comprise an inwardly curved surface, and the non-recessed surface portion can comprise an outwardly curved surface.

In some examples, the medical implant can comprise a skirt attached to the frame, and the skirt can cover at least one strut and the radiopaque marker disposed within the bore of the strut.

In some examples, a medical implant comprises a frame comprising at least one strut having a strut portion with a first concave surface and a second concave surface on opposite sides of the strut portion. The medical implant further comprises a radiopaque marker having an edge surface with a recessed surface portion and a non-recessed surface portion. The radiopaque marker is coupled to the at least one strut with the recessed surface portion positioned in opposing relation to the first concave surface.

In some examples, a medical implant comprises a frame and a radiopaque marker. The frame has a plurality of struts defining a plurality of cells. At least one strut of the plurality of struts has a bore and an eyelet formed therein and a strut portion between the bore and the eyelet. A portion of a wall of the bore forms a first surface of the strut portion and a portion of the wall of the eyelet forms a second surface of the strut portion. The first and second surfaces are opposite sides of the strut portion. The radiopaque marker is disposed within the bore and has an edge surface with a recessed surface portion and a non-recessed surface portion. The recessed surface portion is positioned in opposing relation to the first surface of the strut portion.

In some examples, a medical implant comprises one or more of the components recited in Examples 1-17, Examples 31-54, Examples 66-75, Examples 82-87, and Examples 91-97 below.

A medical assembly can comprise a delivery apparatus, a frame, and a radiopaque marker. In addition to these components, a medical assembly can further comprise one or more of the components disclosed herein.

In some examples, the delivery apparatus can comprise a handle and at least one shaft coupled to the handle.

In some examples, the frame can be coupled to the at least one shaft.

In some examples, the frame can comprise at least one strut having a strut portion with a first concave surface and a second concave surface on opposite sides of the strut portion.

In some examples, the radiopaque marker can have an edge surface with a recessed surface portion and a non-recessed surface portion.

In some examples, the radiopaque marker can be coupled to the at least one strut with the recessed surface portion positioned in opposing relation to the first concave surface.

In some examples, the radiopaque marker can engage the wall of the bore via the non-recessed surface portion.

In some examples, the bore can have a round shape.

In some examples, the frame can comprise a plurality of struts including the at least one strut, and the plurality of struts can define a valve seat for engaging a prosthetic valve.

In some examples, the prosthetic valve can comprise a frame and a valvular structure coupled to the frame.

In some examples, a medical assembly comprises a delivery apparatus, a frame, and a radiopaque marker. The delivery apparatus comprises a handle and at least one shaft coupled to the handle. The frame is coupled to the at least one shaft. The frame comprises at least one strut having a strut portion with a first concave surface and a second concave surface on opposite sides of the strut portion. The radiopaque marker has an edge surface with a recessed surface portion and a non-recessed surface portion. The radiopaque marker is coupled to the at least one strut with the recessed surface portion positioned in opposing relation to the first concave surface.

In some examples, a medical assembly comprises one or more of the components recited in Examples 18-24, Examples 55-65, and Example 88.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

FIG. 1 is an elevation view of an exemplary frame in an expanded state.

FIG. 2A is a detail of a strut of the frame of FIG. 1 illustrating a bore and an eyelet formed in the strut.

FIG. 2B is a top view of the detail of FIG. 2A.

FIG. 3A is a finite element simulation of the strut of FIG. 2A and illustrates strain concentration on an edge portion of the eyelet where the bore is round and contains a round radiopaque marker.

FIG. 3B is a detail of FIG. 3A.

FIG. 4A is a perspective view of an exemplary non-circular radiopaque marker.

FIG. 4B is a top view of the radiopaque marker as depicted in FIG. 4A.

FIG. 4C is a cross-sectional view of the radiopaque marker of FIG. 4A taken along line 4C-4C in FIG. 4B.

FIG. 5 is the detail of FIG. 2A with the radiopaque marker of FIG. 4A mounted within the bore.

FIG. 6A is a top view of another exemplary non-circular radiopaque marker.

FIG. 6B is the top view of the radiopaque marker of FIG. 4B with an aperture added to the radiopaque marker according to another example.

FIG. 7A is a perspective view of another exemplary radiopaque marker.

FIG. 7B is a top view of the radiopaque marker of FIG. 7A.

FIG. 7C is the detail of FIG. 2A with the radiopaque marker of FIG. 7A mounted within the bore.

FIG. 7D is the top view of the radiopaque marker of FIG. 7C with an aperture added to the radiopaque marker according to another example.

FIG. 7E illustrates a bore with a wavy profile in a strut of a frame.

FIG. 7F shows the radiopaque marker of FIG. 7A mounted within the bore in the strut of FIG. 7E.

FIG. 7G is a perspective view of a circular radiopaque marker.

FIG. 7H shows the radiopaque marker of FIG. 7G mounted within the bore in the strut of FIG. 7E.

FIG. 8 is an elevation view of an exemplary docking station including the exemplary frame of FIG. 1 and a skirt attached to the frame.

FIGS. 9A and 9B illustrate attachment of a skirt to the exemplary frame of FIG. 1.

FIG. 10 is a perspective view of an exemplary delivery apparatus.

FIG. 11 illustrates the docking station of FIG. 8 disposed around a distal portion of the delivery apparatus of FIG. 10.

FIG. 12 illustrates encapsulation of the docking station of FIG. 8 within the delivery apparatus of FIG. 10.

FIG. 13 is an elevation view of the docking station of FIG. 8 deployed at an implantation site.

FIG. 14 is the elevation view of the docking station of FIG. 13 with a prosthetic valve mounted in a valve seat of the docking station.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

The subject matter is described with examples. In some cases, as will be recognized by one skilled in the art, the disclosed examples may be practiced without one or more of the disclosed specific details, or may be practiced with other methods, structures, and materials not specifically disclosed herein. All the examples described herein and shown in the drawings may be combined without any restrictions to form any number of combinations, unless the context clearly dictates otherwise, such as if the proposed combination involves elements that are incompatible or mutually exclusive.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. The term “and/or”, when used between the last two elements of a list of elements, means any one or more of the listed elements. The term “or” is generally employed in its broadest sense, that is, as meaning “and/or”, unless the context clearly dictates otherwise.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example”, and “i.e.” means “that is”.

### Overview of the Disclosed Technology

Described herein are docking stations (also referred to herein as docking devices) for implanting a prosthetic valve. In some examples, the docking stations can be implanted within the superior vena cava or the inferior vena cava. In other examples, the docking stations can be implanted in other areas of the anatomy, heart, or vasculature, such as the tricuspid valve, the pulmonary valve, the pulmonary artery, the aortic valve, the aorta, or the mitral valve. In some examples, the docking station includes a frame with a valve seat. A prosthetic valve can be installed in the valve seat. In some examples, radiopaque markers are disposed around the valve seat to assist with deployment of the docking station and mounting of a prosthetic valve in the valve seat. In some examples, the radiopaque markers are mounted within bores formed in selected struts of the frame. In some examples, a radiopaque marker is mounted within a bore by compressing the radiopaque marker such that the radiopaque marker radially expands to engage the bore. In such examples, to reduce strain concentration on the wall of the bore, one of the bore and the radiopaque marker can have a cylindrical surface profile (or circular shape) and the other of the bore and the radiopaque marker has a non-cylindrical surface profile (or non-circular shape), or both the radiopaque marker and the bore can have non-cylindrical surface profiles (or non-circular shapes).

Medical implants (e.g., docking stations or implantable valves) disclosed herein can be radially compressible and expandable between a radially compressed state and a radially expanded state. Thus, the medical implants can be crimped on or retained by an implant delivery apparatus in the radially compressed state during delivery, and then expanded to the radially expanded state once the prosthetic valve reaches the implantation site. It is understood that the medical implants disclosed herein may be used with a variety of implant delivery apparatuses and can be implanted via various delivery procedures, examples of which will be discussed in more detail later.

In some examples, the disclosed docking stations can be implanted within a native heart valve (any of aortic, pulmonary, mitral, or tricuspid valves) or a vessel. For example, in one example, the disclosed docking stations can be implanted within the pulmonary artery for replacing the function of a diseased pulmonary valve, such as disclosed in U.S. Publication No. 2017/0231756, which is incorporated by reference herein. In another example, the disclosed docking stations can be implanted within or at the native mitral valve, such as disclosed in PCT Publication No. WO2020/247907, which is incorporated by reference herein. In another example, the disclosed docking stations can be implanted within the superior or inferior vena cava for replacing the function of a diseased tricuspid valve, such as disclosed in U.S. Publication No. 2019/0000615, which is incorporated herein by reference.

### Examples of the Disclosed Technology

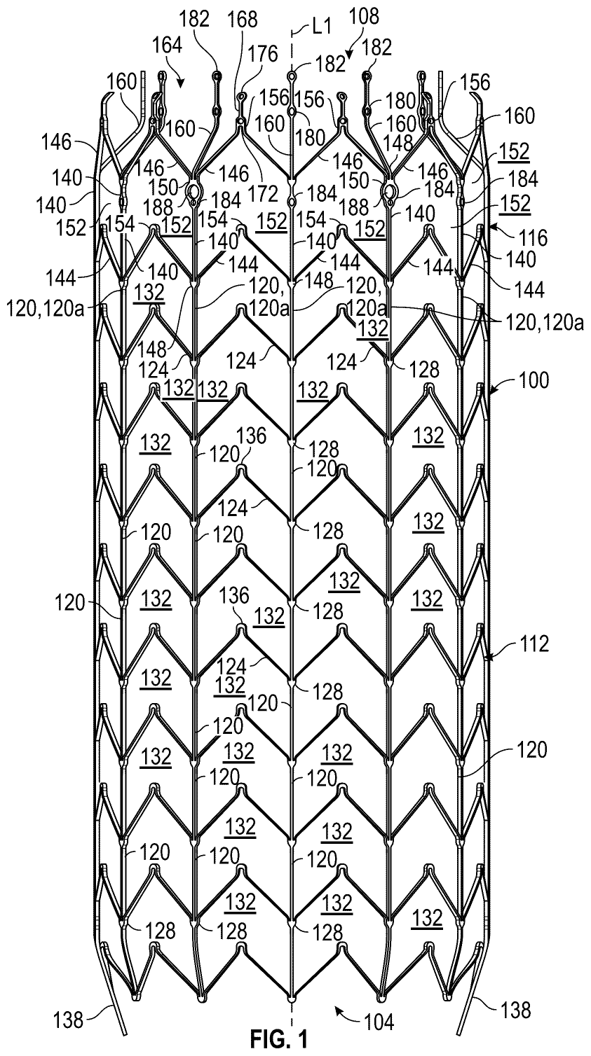

FIG. 1 illustrates an exemplary frame 100 (or stent), according to one example. The frame 100 can be an annular frame and can form a body of a docking station or docking device. The frame 100 has a first end 104 and a second end 108. The frame 100 has a longitudinal axis L1 that extends from the first end 104 to the second end 108. The frame 100 includes a retention portion 112 and a valve seat 116 attached to one end of the retention portion 112. The frame 100 can be deployed at an implantation site within a body (for example, within any of a superior vena cava, tricuspid valve, pulmonary valve, pulmonary artery, mitral valve, aortic valve, or aorta). After deploying the frame 100, a prosthetic valve can be mounted within the valve seat 116.

The frame 100 is shown in an unconstrained, expanded state in FIG. 1. The frame 100 can be compressed for delivery to an implantation site within a body. At the implantation site, the frame 100 can be radially expanded to engage the retention portion 112 and the valve seat 116 with an anatomical surface at the implantation site. The retention portion 112 and the valve seat 116 can exert outward radial forces on the surrounding anatomy to hold the frame 100 in place against the anatomy. In one example, the retention portion 112 can be longer than the valve seat 116 such that the outward radial force applied by the retention portion 112 is spread over a larger area of the anatomy compared to the outward radial force applied by the valve seat 116.

The retention portion 112 of the frame 100 can include first axial struts 120 connected along the longitudinal direction of the frame 100 and spaced about the circumference of the frame. The connected first axial struts 120 extend from the first end 104 of the frame 100 to the valve seat 116. The retention portion 112 can include first angled struts 124 arranged in columns between the first axial struts 120. The first angled struts 124 are connected to the first axial struts 120 at first strut junctions 128. The first angled struts 124 in a column are spaced in the longitudinal direction of the frame 100.

The first angled struts 124 and the first axial struts 120 define rows of first cells 132. The first angled struts 124 have inclined portions forming first apices 136, which are oriented towards the valve seat 116. First cantilever struts 138 can be attached to one or more of the first axial struts 120 at the first end 104 of the frame 100. The first cantilever struts 138 can be used to couple the frame 100 to a delivery apparatus during delivery of the frame 100 to an implantation site.

The valve seat 116 includes second axial struts 140 attached to the upper first axial struts 120 so as to form extensions of the first axial struts 120. The valve seat 116 includes second angled struts 144 and third angled struts 146 extending between the second axial struts 140 and connected to the second axial struts 140 at second strut junctions 148 and third strut junctions 150. The second angled struts 144 and the second axial struts 140 define a row of second cells 152. The second angled struts 144 include inclined portions that define second apices 154 that are oriented towards the second end 108 of the frame 100. The third angled struts 146 include inclined portions that define third apices 156 that are oriented towards the second end 108 of the frame 100.

The frame 100 can include second cantilever struts 160 attached to the third strut junctions 150. The second cantilever struts 160 are spaced about a circumference of the frame 100 by being attached to the third strut junctions 150 that are spaced along the circumference of the frame 100. The second cantilever struts 160 curve radially inwardly, towards a central axis of the frame 100. The second cantilever struts 160 define an opening 164 at the second end 108 of the frame through which a prosthetic valve can be inserted into the valve seat 116.

A prosthetic valve in a compressed state can be inserted into the valve seat 116 through the opening 164 and then radially expanded against the cantilever struts 160, deflecting the cantilever struts 160 radially outwardly. Since the second cantilever struts 160 are biased radially inwardly, the biasing force causes the second cantilever struts 160 to press against the prosthetic valve and center and support the prosthetic valve center within the valve seat 116.

The frame 100 can include third cantilever struts 168 attached to the third apices 156. The third apices 156 can include eyelets 172. The second cantilever struts 168 can include eyelets 176, which can be longitudinally spaced from the eyelets 172 in the third apices 156. The third cantilever struts 168 can include eyelets 180, 182, which can be longitudinally spaced from each other along the respective cantilever strut. The second axial struts 140 can include eyelets 184. The eyelets 172, 180, 182, 184 can be used to attach a skirt to the valve seat 116. For example, a suture can be extended through the eyelets and the skirt to attach the skirt to the valve seat 116.

In some examples, radiopaque markers can be attached to the frame 100 to assist with deployment of the frame 100 at an implantation site and installation of a prosthetic valve into the valve seat 116. In some examples, the radiopaque markers can be disposed circumferentially about the valve seat 116. In some examples, bores 188 can be formed in the second axial struts 140 to hold the radiopaque markers. In the example, the bores 188 are formed in every other second axial strut 140 along the circumference of the frame 100. In other examples, the bores 188 can be formed in all the second axial struts 140 or in fewer than every other second axial strut 140. In the illustrated example, each second axial strut 140 including a bore 188 can also include one of the eyelets 184.

The frame 100 can be made of any of various suitable plastically-expandable materials (for example, stainless steel, etc.) or self-expanding materials (for example, Nitinol) as known in the art. When constructed of a plastically-expandable material, the frame 100 (and thus the docking station) can be crimped to a radially compressed state on a delivery catheter and then expanded inside a patient by an inflatable balloon or equivalent expansion mechanism. When constructed of a self-expandable material, the frame 100 (and thus the docking station) can be crimped to a radially compressed state and restrained in the compressed state by insertion into a sheath or equivalent mechanism of a delivery catheter. Once inside the body, the docking station can be advanced from the delivery sheath, which allows the docking station to expand to its functional size.

Suitable plastically-expandable materials that can be used to form the frames disclosed herein (for example, the frame 100) include metal alloys, polymers, or combinations thereof. Example metal alloys can comprise one or more of the following: nickel, cobalt, chromium, molybdenum, titanium, or other biocompatible metal. In some examples, the frame 100 can comprise stainless steel. In some examples, the frame 100 can comprise cobalt-chromium. In some examples, the frame 100 can comprise nickel-cobalt-chromium. In some examples, the frame 100 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Further details regarding docking stations and docking devices can be found, for example, in U.S. Patent Application Publication No. 2019/0000615, which is incorporated herein by reference.

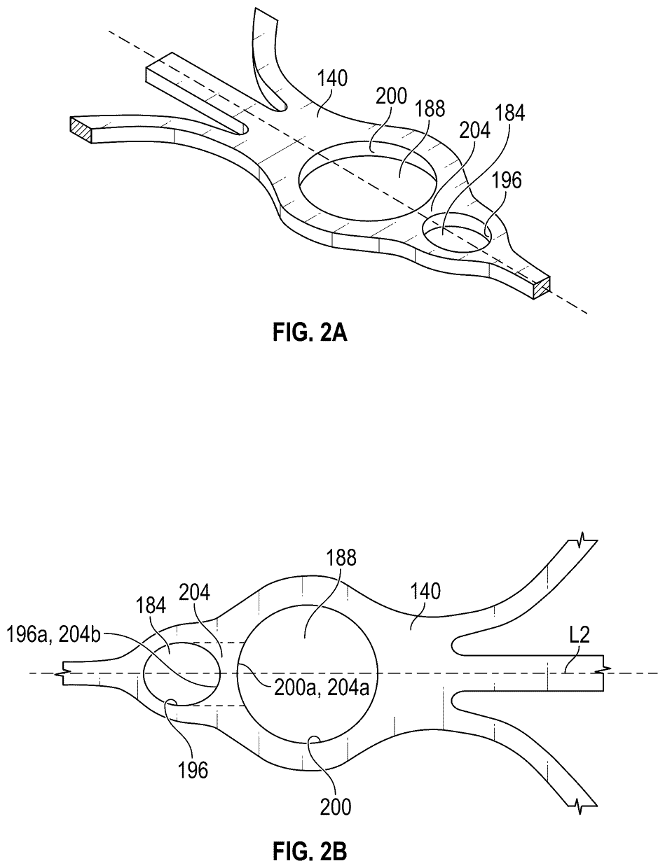

FIGS. 2A and 2B illustrate a portion of the second axial strut 140 including an eyelet 184 and a bore 188. The eyelet 184 is a hole defined by a wall 196. The bore 188 is a hole defined by a wall 200. The second axial strut 140 has a strut portion 204 between the eyelet 184 and the bore 188. Portion 196a of the bore wall 196 forms a first surface 204a (or first side) of the strut portion 204. Portion 200a of the eyelet wall 200 forms a second surface 204b (or second side) of the strut portion. The first and second surfaces 204a, 204b are on opposite sides of the strut portion 204. In one example, the eyelet 184 and the bore 188 can have round shapes (e.g., circular shape or oval shape). In this case, the first and second surfaces 204a, 204b are concave surfaces. The second axial strut 140 can be in the form of an elongated plate having a longitudinal axis L2, with the eyelet 184 and the bore 188 spaced along the longitudinal axis L2 and extending through the thickness of the plate.

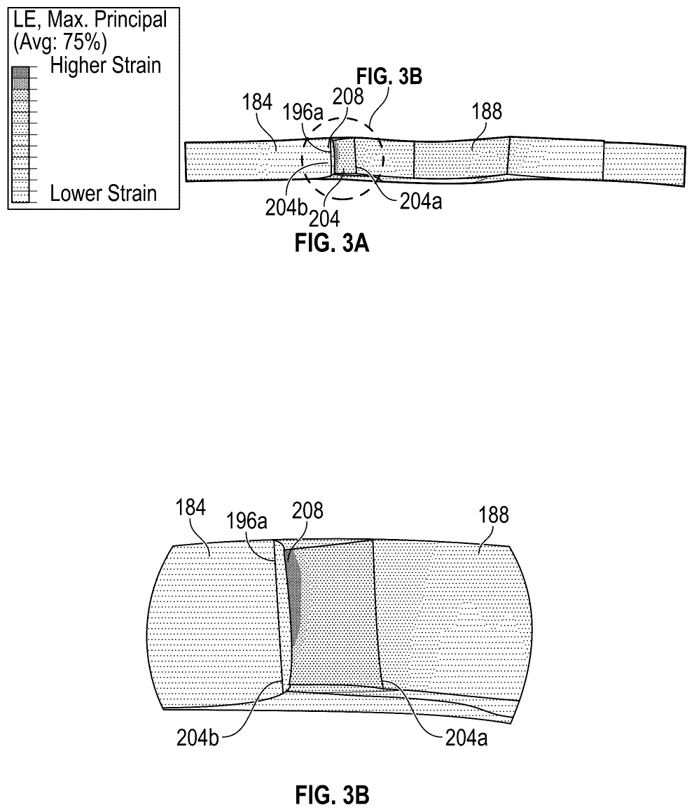

FIGS. 3A and 3B show a result of a finite element analysis of a second axial strut 140 for an example where the bore 188 is circular and a radiopaque marker assembled into the bore 188 is circular (or disc-shaped). In this example, the radiopaque marker initially has a thickness (or height) that is greater than a height of the wall of the bore 188. The radiopaque marker is pressed (or flattened) while being assembled into the circular bore. As the radiopaque marker is pressed down, the radiopaque marker becomes thinner and expands radially outward, exerting outward radial pressure on the wall of the bore. This outward radial pressure radially expands the wall of the bore, resulting in tension strains along portions of the strut adjacent to the wall of the bore. In the analysis shown in FIGS. 3A and 3B, high strain concentration can be observed near the second surface 204b of the strut portion 204 facing the eyelet 184, as indicated at 208, as a result of the outward radial pressure exerted by the radiopaque marker on the first surface 204a of the strut portion 204. High strain concentration in the strut can result in cracks in the strut. In some examples herein, non-circular radiopaque markers are disclosed that when mounted in a bore can avoid high strain concentration in strut portions around the bore, such as the strut portion between the bore and the eyelet.

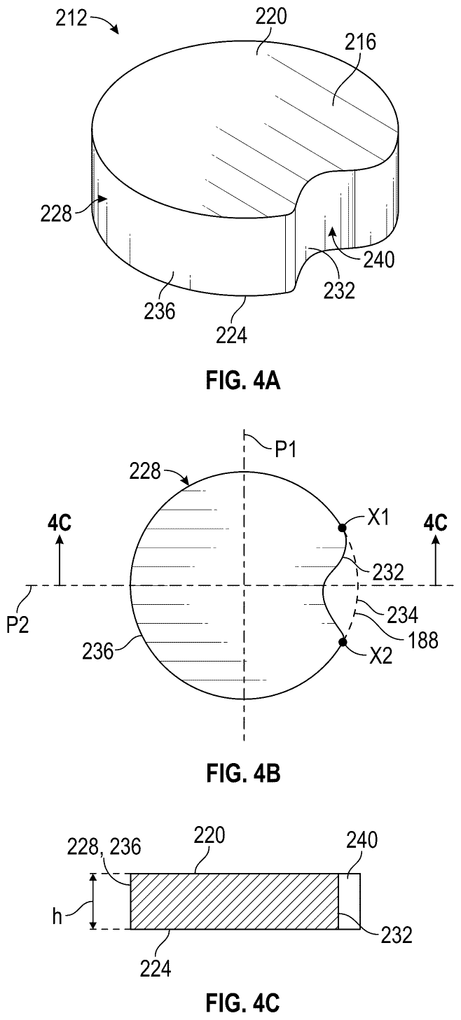

FIGS. 4A-4C show an example of a non-circular radiopaque marker 212. The radiopaque marker 212 includes a marker body 216 having opposite major surfaces 220, 224 (top and bottom surfaces in the figure) and an edge surface 228 extending between the major surfaces 220, 224 and circumscribing the marker body 216.

In one example, the edge surface 228 can be a vertical wall between the major surfaces 220, 224 (as depicted in FIGS. 4A-4C). In another example, the edge surface 228 can be an inclined wall (for example, such that the marker body 216 has a tapered shape in a direction from the major surface 220 to the major surface 224, or vice versa).

In one example, the major surfaces 220, 224 can be planar surfaces (as depicted in FIGS. 4A and 4C). In other examples, the major surfaces 220, 224 can be curved surfaces (for example, convex or concave surfaces) or beveled surfaces.

In one example, the height h (depicted in FIG. 4C) of the marker body 216 (which is also the height of the edge surface 228) can be selected to match the height of a bore 188 in a strut. In another example, the height h can be taller or shorter than the height of a bore 188 in a strut.

The edge surface 228 includes a recessed surface portion 232 and a non-recessed surface portion 236 that together define a non-cylindrical surface profile. The recessed surface portion 232 can be an inwardly curved surface portion, and the non-recessed surface portion 236 can be an outwardly curved edge surface portion. The surface portions 232, 236 are contiguous in that the ends of the recessed surface portion 232 turn (or curve) into the non-recessed surface portion 236. The points X1 and X2 in FIG. 4B indicate where the recessed surface portion 232 curves into (or is joined to) the non-recessed surface portion 236. In one example, the non-recessed surface portion 236 can be a portion of a cylindrical wall (the dashed line 234 shows a full cylindrical wall for comparative purposes.)

In one example, the radiopaque marker 212 can be asymmetric about a plane that is transverse to the major surfaces 220, 224 (for example, a plane P1 in FIG. 4B). In another example, the radiopaque marker 212 can be asymmetric about a plane that is transverse to the two major surfaces 220, 224 and intersecting the recessed surface portion 232 and the non-recessed surface portion 236 (for example, plane P2 in FIG. 4B).

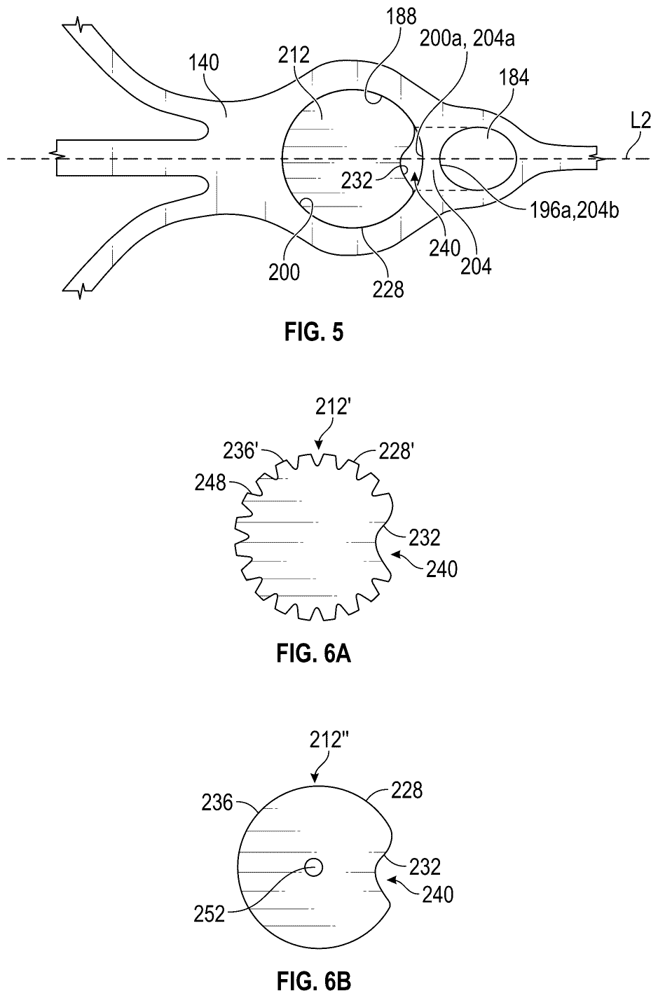

FIG. 5 illustrates the radiopaque marker 212 mounted within a bore 188 formed in a second axial strut 140 of the valve seat (116 in FIG. 1). The radiopaque marker 212 can be mounted in the bore 188 using any suitable method (such as by adhesive, press fit, welding, snap fit, fasteners, such as a suture, or various combinations thereof). In some examples, the marker 212 can be inserted into the bore 188, and a compressive force can be applied to the opposing surfaces 220, 224 of the marker 212 that causes the initial height h of the marker to decrease while the marker expands radially against the inner surface of the bore 188, thereby securing the marker within the bore. In such examples, the initial height of the marker can be slightly greater than the height of the bore 188. After compression, the height h of the marker 212 can be the same as or less than the height of the bore. The initial maximum diameter of the marker (measured along plane P1 in the example of FIGS. 4A-5) before compression can be slightly smaller than the inner diameter of the bore 188 to allow the marker to be easily inserted into the bore 188.

The radiopaque markers 212 can comprise any material or combination of materials that are radiopaque or increase the radiopacity of the valve seat. For example, the radiopaque markers 212 can comprise barium sulfate, bismuth, tungsten, tantalum, platinum-iridium, gold or any other material which is opaque to fluoroscopy, X-rays, or similar radiation or any combination thereof. In other examples, the radiopaque markers 212 can be formed from a first material that has a relatively low radiopacity and a second material that has a relatively high radiopacity (the second material can be for example, barium sulfate, bismuth, tungsten, tantalum, platinum-iridium, or gold). The second material can be in the form of particles that are mixed or dispersed within the first material.

The mounting of the radiopaque marker 212 within the bore 188 can be such that the recessed surface portion 232 formed in the edge surface 228 of the radiopaque marker 212 is in opposing relation to the first surface 204a of the strut portion 204 (or bore wall portion 200a) and such that the non-recessed surface portion 236 engages the wall 200 of the bore 188 except for the bore wall portion 200a. A pocket 240 is formed between the recessed surface portion 232 and the bore wall portion 200a (or first surface 204a of the strut portion 204). The width of the recessed surface portion 232 can be selected such that the edge surface 228 does not contact the first surface 204a of the strut portion 204 when the radiopaque marker 212 is mounted within the bore 188.

The recessed surface portion 232 can be positioned along the longitudinal axis L2 of the strut. In some examples, the recessed surface portion 232 can be asymmetrical about the longitudinal axis L2 (as depicted in FIG. 5). In other examples, the recessed surface portion 232 can be symmetrical about the longitudinal axis L2. The profile of the non-recessed surface portion 236 can match that of the wall 200 of the bore 188 such that the non-recessed surface portion 236 engages the wall 200 of the bore 188 except for the bore wall portion 200a (as depicted in FIG. 5).

The non-recessed surface portion 236 can be a smooth surface as depicted in FIGS. 4A-4C and 5. In another example, as depicted in FIG. 6A, the non-recessed surface portion 236′ of the radiopaque marker 212′ can include grooves 248 (or channels). The grooves 248 can minimize the contact area between the non-recessed surface portion 236′ and the wall 200 of the bore 188 when the radiopaque marker 212′ is mounted within the bore 188.

In some examples, as depicted in FIG. 6B, the radiopaque marker 212″ can be provided with one or more apertures 252 that can be used for suturing the radiopaque marker 212″ to the frame or to a skirt attached to the frame. For example, while suturing the skirt to the frame, the suture can be passed through the apertures 252 as well. Suturing can be used in addition to, or in lieu of, other methods of securing the radiopaque marker to the bore.

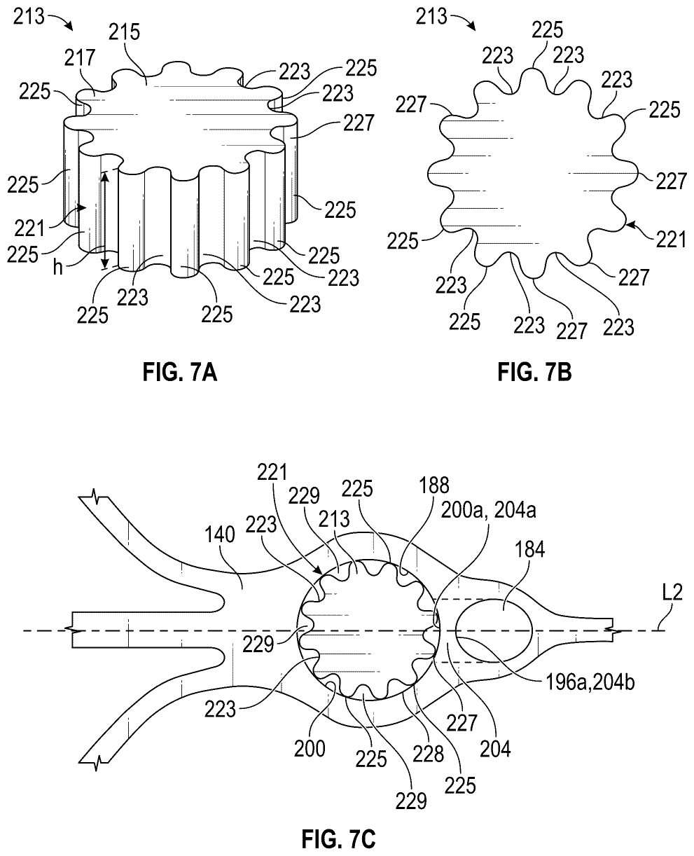

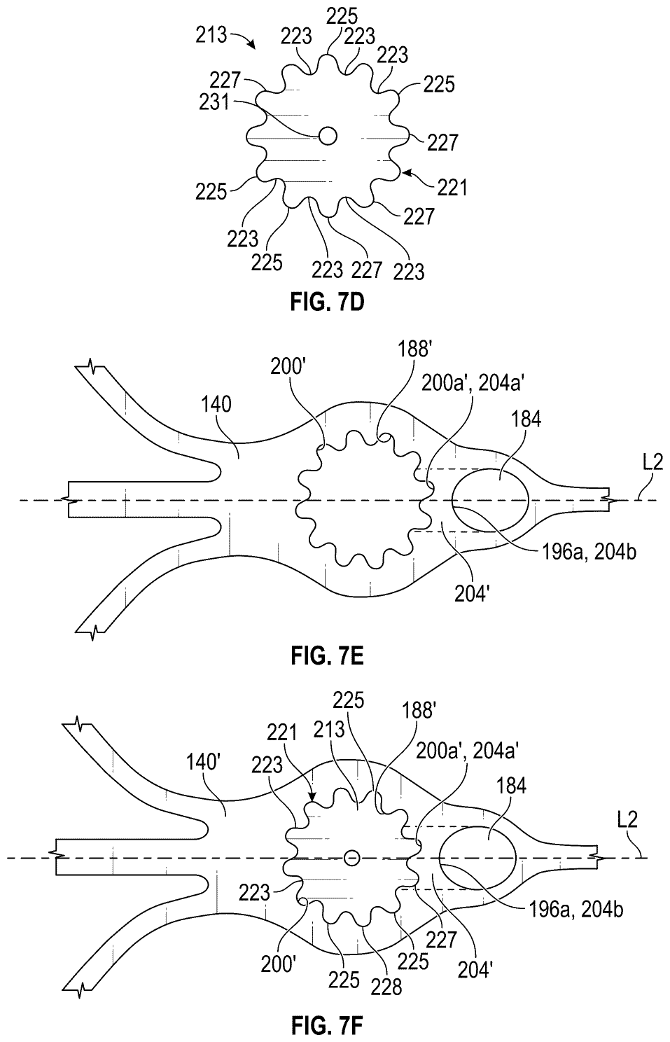

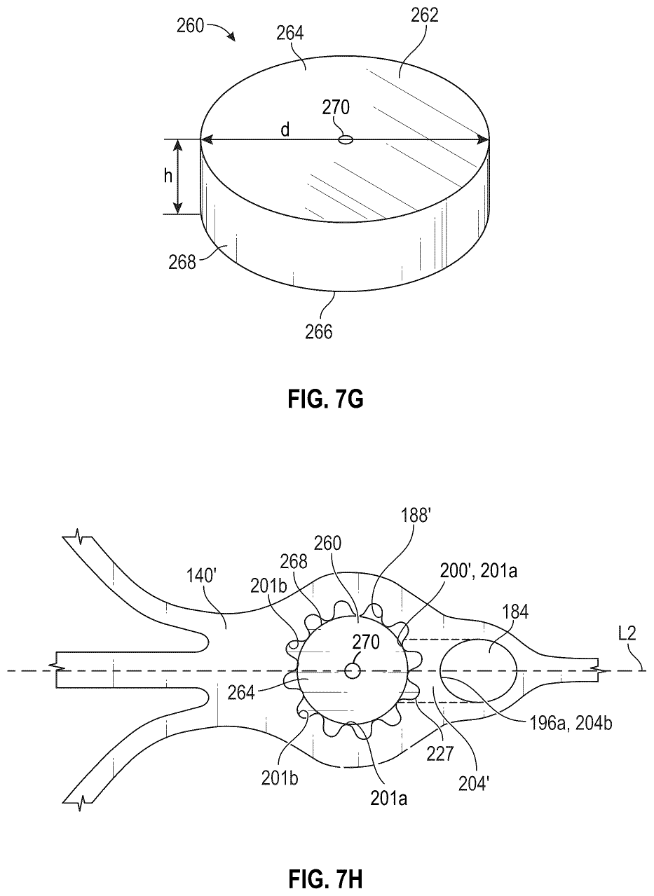

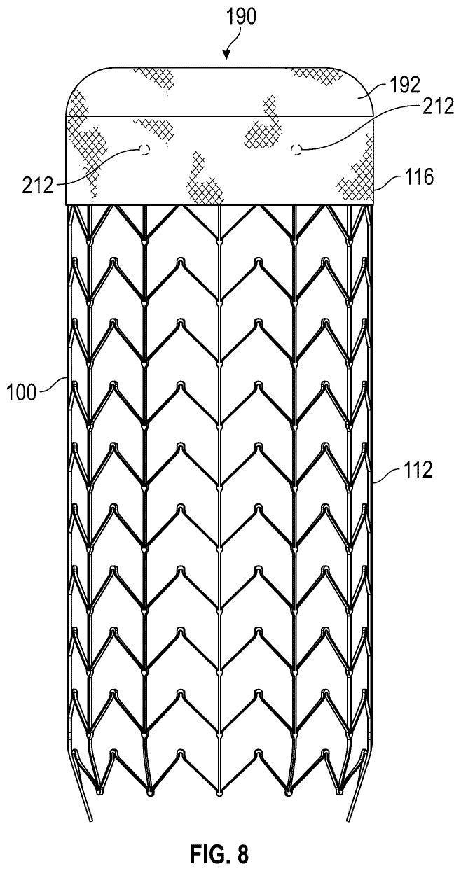

FIGS. 7A and 7B illustrate an exemplary non-circular radiopaque marker 213, according to another example. The radiopaque marker 213 includes a marker body 215 having opposite major surfaces 217, 219 (top and bottom surfaces in the figure) and an edge surface 221 extending between the major surfaces 217, 219 and circumscribing the marker body 215.

In one example, the major surfaces 217, 219 can be planar surfaces (as shown in FIG. 7A). In other examples, the major surfaces 217, 219 can be curved surfaces (for example, convex or concave surfaces) or beveled surfaces.

In one example, the height h of the marker body 215 (or the height of the edge surface 221) can be selected to match the height of a bore in a strut. In another example, the height h of the marker body 215 can be taller or shorter than the height of a bore in a strut.

The edge surface 221 includes a series of recessed surface portions 223 (forming recesses) spaced about the circumference of the marker body 215 and a series of non-recessed surface portions 225 (forming radial projections) spaced about the circumference of the marker body 215. The recessed surface portions 223 and the non-recessed surface portion 225 can be in alternating arrangement about the circumference of the marker body 215 and can be contiguous to form a continuous edge surface 221. The recessed surface portions 223 and non-recessed surface portions 225 together define a non-cylindrical surface profile. The edge surface 221 can be a wavy or undulating surface (as illustrated in FIGS. 7A and 7B). The recessed surface portions 223 can be inwardly curved surface portions, and the non-recessed surface portions 225 can be outwardly curved surface portions. The non-recessed surface portions 225 can have tip portions 227 for engaging the wall of a bore in a strut. In one example, the tip portions 227 are rounded.

FIG. 7C shows the non-circular radiopaque marker 213 mounted within a bore 188 formed in a second axial strut 140 of the valve seat (116 in FIG. 1). The radiopaque marker 213 can be mounted in the bore 188 using any of the methods previously described for the radiopaque marker 212. The radiopaque marker 213 can comprise any of the materials previously described for the radiopaque marker 212.

In the illustrated example, the bore 188 has a round geometry. The mounting of the radiopaque marker 213 within the bore 188 can be such that tip portions 227 of the non-recessed surface portions 225 of the edge surface 221 engage the wall 200 of the bore 188 and a series of pockets 229 are formed between the recessed surface portions 223 and the wall 200 of the bore 188. Since only the tip portions 227 of the non-recessed surface portions 225 contact the first surface 204a (bore side surface) of the strut portion 204, the contact area between the radiopaque marker 213 and the first surface 204a (bore side surface) of the strut portion 204 is minimized.

Similar to the example illustrated in FIG. 6B, the radiopaque marker 213 can be provided with one or more apertures (for example, aperture 231 as shown in FIG. 7D) that can be used for suturing the radiopaque marker 213 to the frame or to a skirt attached to the frame. For example, while suturing the skirt to the frame, the suture can be passed through the aperture 231 as well.

When the bore 188 has a round shape, strain on the second surface 204b (eyelet side surface) of the strut portion 204 (for example, due to bending of the strut 204) can be reduced by minimizing the contact area between a radiopaque marker mounted within the bore 188 and the first surface 204a (bore side surface) of the strut portion 204. Advantageously, the non-circular radiopaque markers described herein are configured to be mounted within a round bore in a manner that reduces strain on the second surface 204b (eyelet side) of the strut portion 204. For example, the radiopaque markers 212 illustrated in FIG. 4A (and its variants illustrated in FIGS. 6A and 6B) can be mounted within the bore 188 with the recessed surface portion 232 in opposing relation to the first surface 204a (bore side) of the strut portion 204, thereby avoiding contact with the first surface 204a (bore side) of the strut portion 204 such that strain in the second surface 204b is reduced when the strut is subjected to bending. In the case of the radiopaque marker 215 that can contact the first surface 204a (bore side) of the strut portion 204, the contact is minimized by allowing the radiopaque marker 215 to contact the first surface 204a only via tip portions 227 of the recessed surface portions 223.

In other examples, the non-circular radiopaque markers can be mounted in bores that do not have a round geometry. FIG. 7E illustrates an example bore 188′ in a strut 140′. The bore 188′ is connected to an eyelet 184 by a strut portion 204′ such that a portion 200a′ of the bore wall 200′ forms a first surface 204a′ of the strut portion 204′ and a portion 196a of the eyelet 184 forms a second surface 204b′ of the strut portion 204′. The wall 200′ of the bore 188′ has a wavy profile that is complementary to that of the edge surface 221 (shown in FIGS. 7A, 7B, and 7C) of the radiopaque marker 213.

FIG. 7F shows the radiopaque marker 213 mounted in the bore 188′. As illustrated, there can be contact between the entire edge surface 221 of the radiopaque marker 213 and the bore wall 200′ (including the wall portion 200′/first surface 204a′ of the strut portion 204′) such that the contact area between the radiopaque marker 213 and the bore wall 200′ is maximized. In this example, the wavy profile of the bore wall 200′ may avoid high strain in the second surface 204b (eyelet side) of the strut portion 204′ such that it is now advantageous to increase the contact area between the radiopaque marker 213 and the bore wall 200′.

FIG. 7G illustrates a circular radiopaque marker 260, according to one example. The radiopaque marker 260 includes a marker body 262 having opposite major surfaces 264, 266 (top and bottom surfaces in the figure) and an edge surface 268 extending between the major surfaces 264, 266 and circumscribing the marker body 262. In one example, the major surfaces 264, 266 can be planar surfaces (as shown in FIG. 7G). In other examples, the major surfaces 264, 266 can be curved surfaces (for example, convex or concave surfaces) or beveled surfaces. The edge surface 268 has a cylindrical surface profile.

The circular radiopaque marker 260 can be mounted within a bore having a wall with a non-cylindrical profile (which can also be referred to as a non-circular bore). FIG. 7H illustrates an example where the circular radiopaque marker 260 is mounted within the bore 188′ (previously shown in FIGS. 7E and 7F). The wall 200′ of the bore 188′ has a wavy profile including radial projections 201a spaced along a circumference of the bore 188′ and recesses 201b between the radial projections 201a. The radial projections 201a extend toward a center of the bore 188′. The cylindrical edge surface 268 of the circular radiopaque marker 260 frictionally engages the radial projections 201a of the wall 200′ of the bore 188′.

A method of mounting the circular radiopaque marker 260 within the bore 188′ can include inserting the circular radiopaque marker 260 within the central opening defined by the radial projections 201a of the wall 200′ of the bore 188′. A compressive force can be applied to the opposing surfaces 264, 266 of the marker that causes the height h (shown in FIG. 7G) of the marker to decrease while the marker expands radially against the radial projections 201a, thereby frictionally engaging the marker edge surface 268 with the bore wall radial projections 201a. The recesses 201b between the radial projections 201a can accommodate any excess radial expansion of the circular radiopaque marker 260 (that is, portions of the edge surface 268 can flow into the recesses 201b during radial expansion of the marker) such that tension strain concentrations in the wall 200′ due to radial expansion of the marker is reduced.

In some examples, the initial height h (shown in FIG. 7G) of the circular radiopaque marker 260 can be slightly greater than the height of the bore 188′. After compressing the circular radiopaque marker 260 as described above, the height of the circular radiopaque marker 260 can be the same or less than the height of the bore 188′. The initial diameter d (shown in FIG. 7G) of the marker before compression can be slightly smaller than the diameter of the central opening formed by the radial projections 201a of the wall 200′ of the bore 188′ to allow the circular radiopaque marker 260 to be easily inserted into the bore 188′.

In some examples, the radiopaque marker 260 can be provided with one or more apertures 270 (shown in FIG. 7G) that can be used for suturing the marker to a frame or to a skirt attached to the frame. Suturing of the marker can be used in addition to press-fitting the radiopaque marker 260 into the bore 188′.

FIG. 8 illustrates an exemplary docking station 190 including the frame 100 with the radiopaque markers 212 mounted in the bores 188 (as illustrated in FIG. 5) in the valve seat 116 and with a skirt 192 disposed around the valve seat 116.

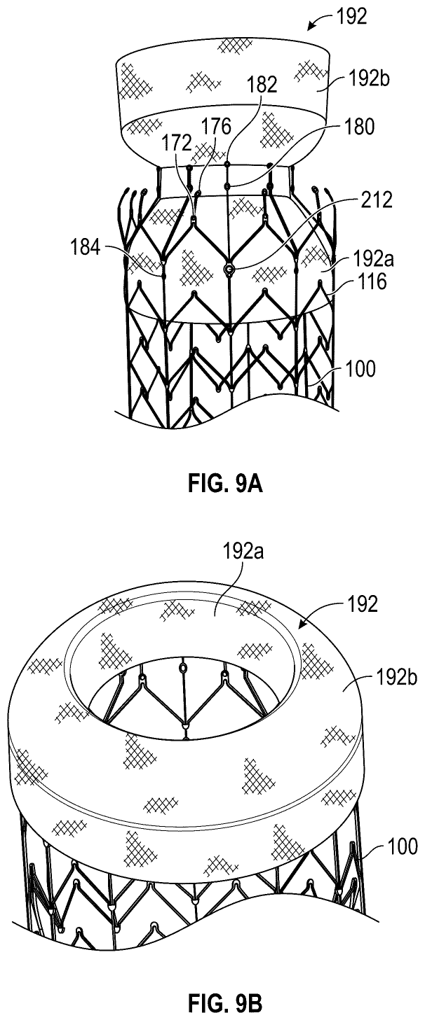

FIGS. 9A and 9B illustrate attachment of the skirt 192 to the frame 100. For the example illustrated in FIGS. 9A and 9B, the radiopaque markers 212 are mounted within the bores 188 in the frame 100 (any of the radiopaque markers disclosed herein can be mounted within the bores 188). FIG. 9A shows an inner skirt portion 192a of the skirt 192 arranged on the inside of a portion of the frame 100 including the valve seat 116. FIG. 9B shows an outer skirt portion 192b of the skirt 192 arranged on the outside of a portion of the frame 100 including the valve seat 116. In the example, the radiopaque markers are encapsulated by the skirt (for example, between the inner skirt portion 192a and outer skirt portion 192b of the skirt.) The skirt 192 covers the second cells (152 in FIG. 1) and the struts that form the valve seat 116. The skirt 192 can be attached to the frame 100, for example, by suturing and using the eyelets 172, 180, 182, 184 (not all the eyelets are labeled in FIG. 9A).

In the illustrated example, the skirt 192 extends over and covers the second end 108 of the frame 100. In other examples, the inner skirt portion 192a and the outer skirt portion 192b can be provided as separate skirts mounted respectively on the inside and outside of the frame. In other examples, the skirt 192 may only cover one side of the frame 100 (for example, the inner side or the outer side of the frame 100).

When the frame 100 is implanted within an anatomy, the skirt 192 can seal against the tissue of the anatomy and help to reduce paravalvular leakage past the prosthetic valve mounted within the valve seat 116. The skirt 192 can be wholly or partly formed of any suitable biological material, synthetic material (for example, any of various polymers), or combinations thereof. In some examples, the skirt 192 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt 192 can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt 192 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt 192 can comprise a sponge material or foam, such as polyurethane foam. In some examples, the skirt 192 can comprise natural tissue, such as pericardium (for example, bovine pericardium, porcine pericardium, equine pericardium, or pericardium from other sources). Further details regarding the use of skirts or sealing members in prosthetic valves can be found, for example, in U.S. Patent Publication No. 2020/0352711, the relevant disclosure of which is incorporated herein by reference.

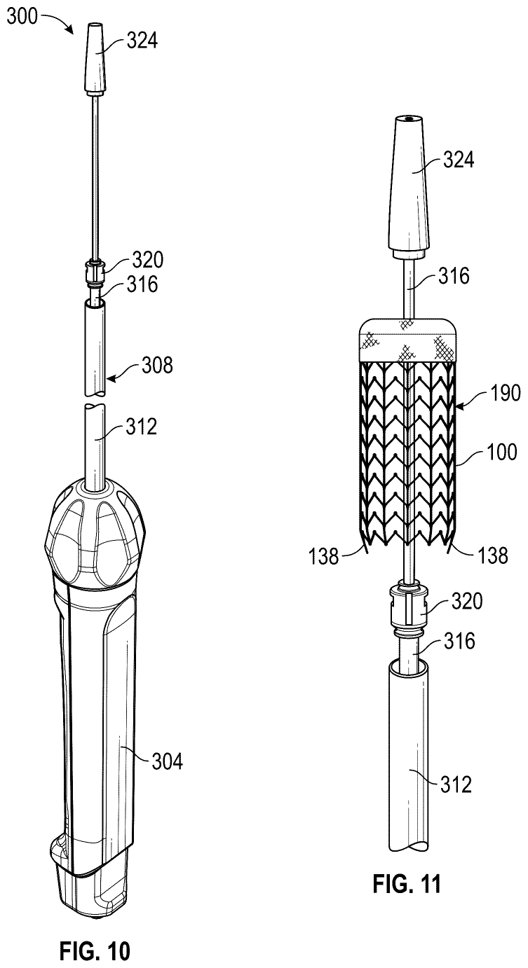

FIG. 10 illustrates an exemplary delivery apparatus 300 that can be used to deliver the docking station (190 in FIG. 8) to an implantation site. The delivery apparatus 300 generally includes a handle 304 and a delivery catheter 308 extending distally from the handle 304. The delivery catheter 308 includes an outer shaft 312 and an inner shaft 316 extending through a lumen of the outer shaft 312. The docking station 190 can be disposed around the inner shaft 316. A frame connector 320 on the inner shaft 316 can include recesses to receive the connection struts (138 in FIG. 1) at the end of the frame (100 in FIG. 1) of the docking station 190. A nosecone 324 can be attached to a distal end of the inner shaft 316. The nosecone 324 can include a central opening for passage of a guidewire. The guidewire can pass through the nosecone 324 into the inner shaft 316 during advancing of the delivery catheter 308 into a patient's vasculature.

The handle 304 can be operated to move the outer shaft 312 along the inner shaft 316, generally between an extended position and a retracted position. The handle 304 can be extended to slide the outer shaft 312 over the frame connector 320 and over the docking station coupled to the frame connector 320 to encapsulate the docking station within the outer shaft 312. As the outer shaft 312 slides over the docking station, the outer shaft 312 can compress the docking station such that the docking station is encapsulated within the outer shaft 312 in the compressed state. Alternatively, the docking station can be compressed and held in the compressed state with a removable band prior to extending the outer shaft 312 over the docking station. In the fully extended position of the outer shaft 312, a distal end of the outer shaft 312 can abut a proximal end of the nosecone 324 such that there are no gaps in the delivery assembly. Further details regarding delivery apparatus for a docking station or docking device can be found in, for example, U.S. Provisional Application No. 63/154,956, the relevant disclosure of which is incorporated herein by reference.

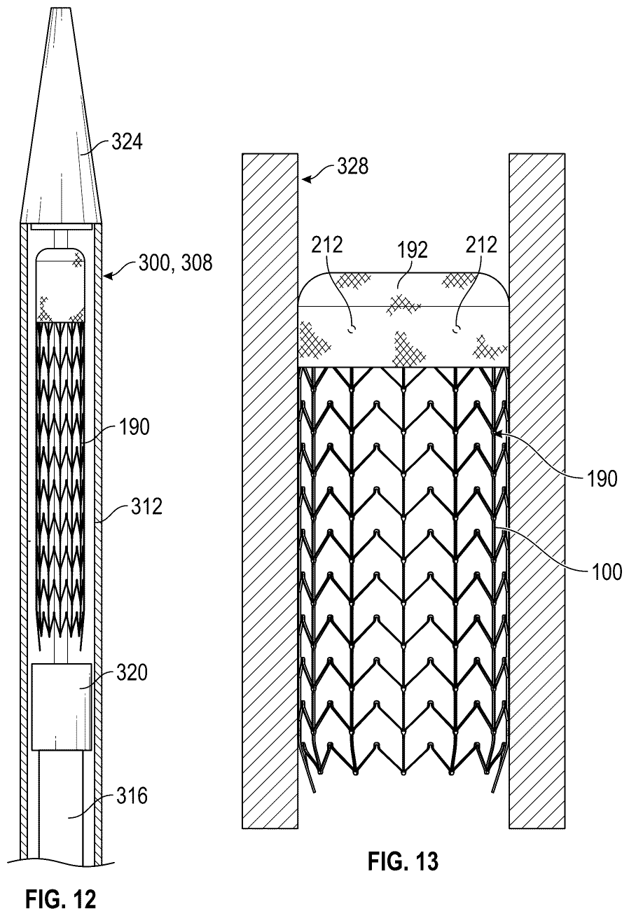

A method of implanting the docking station 190 at an implantation site within an anatomy can include retracting the outer shaft 312 by the handle 304 of the delivery apparatus 300 to allow loading of the docking station 190 onto the inner shaft. The method can include positioning the docking station 190 around the inner shaft 316, as shown in FIG. 11, and engaging the connection struts 138 of the frame 100 of the docking station 190 with the frame connector 320. The method can include compressing the docking station 190 and extending the outer shaft 312 by the handle 304 to encapsulate the docking station 190 in the compressed state, as illustrated in FIG. 12. The extension of the outer shaft 312 can position the distal end of the outer shaft 312 against the proximal end of the nosecone 324, as illustrated in FIG. 12. The method can include inserting the delivery apparatus 300, from the nosecone 324, into a patient's vasculature and advancing the delivery catheter (for example, over a guidewire) through the patient's vasculature to the implantation site.

At the implantation site, the method can include retracting the outer shaft 312 by the handle 304 of the delivery apparatus to expose the docking station 190. The radiopaque markers 212 on the frame 100 of the docking station 190 can assist in positioning the docking station 190 at the implantation site under fluoroscopy. In cases where the docking station 190 is self-expandable, the docking station 190 can gradually emerge from the outer shaft 312 and gradually expand from the compressed state as the outer shaft 312 is retracted. When the outer shaft 312 is sufficiently retracted, the connection struts 138 of the frame 100 can disengage from the frame connector 320. Once the docking station 190 is disengaged from the frame connector 320, the docking station 190 can radially expand to engage the anatomy.

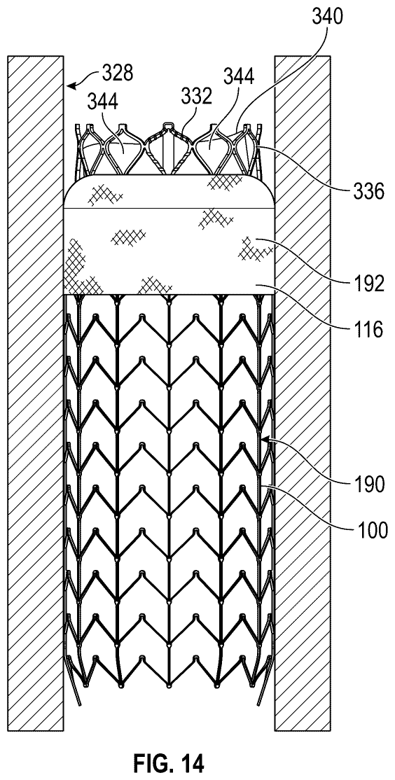

FIG. 13 illustrates the docking station 190 in a deployed state at an implantation site 328 (for example, a heart valve or lumen). After deploying the docking station 190 at the implantation site 328, a prosthetic valve can be delivered to the implantation site. FIG. 14 illustrates a prosthetic valve 332 mounted within the valve seat 116 of the docking station 190. The radiopaque markers 212 on the frame 100, which in this example are behind the skirt 192, can be used to guide mounting of the prosthetic valve 332 within the valve seat.

In one example, the prosthetic valve 332 can be configured to replace a native heart valve (e.g., aortic, mitral, pulmonary, and/or tricuspid valves). In one example, the prosthetic valve 332 can include a frame 336 and a valvular structure 340 disposed within and attached to the frame 336. The valvular structure 340 can include one or more leaflets 344 that cycle between open and closed states during the diastolic and systolic phases of the heart. The frame 336 can be made of any of the frame materials described for the frame 100 of the docking station 190. The leaflets 344 can be made in whole or in part from pericardial tissue (for example, bovine pericardial tissue), biocompatible synthetic materials, or various other suitable natural or synthetic materials known in the art. Further details regarding prosthetic valves are disclosed in U.S. Pat. Nos. 8,652,202 and 9,393,110 and U.S. Publication Nos. 2018/0028310 and 2019/0365530, which are incorporated herein by reference.

The marker and bore configurations shown in FIGS. 2A-7 and described above can be implanted in any of various medical implants having a frame, including, without limitation, prosthetic valves, stents, stent grafts, and annuloplasty rings. For example, a prosthetic valve (or another type of medical implant) according to the present disclosure can include a frame having one or more bores 188 (which can be formed in one or more respective strut of the frame) and one or more markers 212, 212′, or 212″ disposed in respective bore(s). Each bore can be adjacent an eyelet (such as eyelet 184), which can receive a suture for securing a skirt, a prosthetic leaflet, or another component of the prosthetic valve to the frame. For example, the prosthetic valve can have an inner skirt and/or an outer skirt that is connected to the frame with sutures extending through the eyelets that are adjacent bores in which the markers are disposed.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Delivery Techniques

For implanting a medical implant (e.g., a docking station or a prosthetic valve) within the native aortic valve via a transfemoral delivery approach, the medical implant is mounted in a radially compressed state along the distal end portion of a delivery apparatus. The medical implant and the distal end portion of the delivery apparatus are inserted into a femoral artery and are advanced into and through the descending aorta, around the aortic arch, and through the ascending aorta. The medical implant is positioned within the native aortic valve and radially expanded (e.g., by inflating a balloon, actuating one or more actuators of the delivery apparatus, or deploying the medical implant from a sheath to allow the medical implant to self-expand). Alternatively, a medical implant can be implanted within the native aortic valve in a transapical procedure, whereby the medical implant (on the distal end portion of the delivery apparatus) is introduced into the left ventricle through a surgical opening in the chest and the apex of the heart and the medical implant is positioned within the native aortic valve. Alternatively, in a transaortic procedure, a medical implant (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a medical implant (e.g., a docking station or a prosthetic valve) within the native mitral valve via a transseptal delivery approach, the medical implant is mounted in a radially compressed state along the distal end portion of a delivery apparatus. The medical implant and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a medical implant can be implanted within the native mitral valve in a transapical procedure, whereby the medical implant (on the distal end portion of the delivery apparatus) is introduced into the left ventricle through a surgical opening in the chest and the apex of the heart and the medical implant is positioned within the native mitral valve.

For implanting a medical implant (e.g., a docking station or a prosthetic valve) within the native tricuspid valve, the medical implant is mounted in a radially compressed state along the distal end portion of a delivery apparatus. The medical implant and the distal end portion of the delivery apparatus are inserted into a femoral vein and are advanced into and through the inferior vena cava, and into the right atrium, and the medical implant is positioned within the native tricuspid valve. A similar approach can be used for implanting the medical implant in the inferior vena cava, except that the implant is not advanced into the right atrium and instead is deployed when it reaches a desired location within the superior vena cava. For implanting the implant in the superior vena cava, the implant is advanced from the inferior vena cava into the superior vena cava and then deployed. A similar approach can be used for implanting the medical implant within the native pulmonary valve or the pulmonary artery, except that the medical implant is advanced through the native tricuspid valve into the right ventricle and toward the pulmonary valve/pulmonary artery.

Another delivery approach is a transatrial approach whereby a medical implant (e.g., a docking station or a prosthetic valve) (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a medical implant (e.g., a docking station or a prosthetic valve) (on the distal end portion of the delivery apparatus) is inserted through an incision in the chest and an incision made through the wall of the right ventricle (typically at or near the base of the heart) for implanting the medical implant within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the medical implants disclosed herein can be implanted using any of various delivery procedures and delivery devices known in the art.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### ADDITIONAL EXAMPLES

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application. Example 1: A medical implant comprising: a frame comprising at least one strut having a strut portion with a first concave surface and a second concave surface on opposite sides of the strut portion; and a radiopaque marker having an edge surface with a recessed surface portion and a non-recessed surface portion, the radiopaque marker coupled to the at least one strut with the recessed surface portion positioned in opposing relation to the first concave surface.

Example 2: The medical implant according to any example herein, particularly Example 1, wherein the at least one strut includes a bore adjacent to the strut portion, wherein a portion of a wall of the bore forms the first concave surface, and wherein the radiopaque marker is disposed within the bore.

Example 3: The medical implant according to any example herein, particularly Example 2, wherein the non-recessed surface portion engages the wall of the bore.

Example 4: The medical implant of any example herein, particularly any one of Examples 2-3, wherein a longitudinal axis of the strut extends through the first and second concave surfaces, and wherein the radiopaque marker is asymmetric about the longitudinal axis of the strut.

Example 5: The medical implant of any example herein, particularly any one of Examples 2-4, wherein the at least one strut includes an eyelet adjacent to the strut portion, and wherein a portion of a wall of the eyelet forms the second concave surface.

Example 6: The medical implant of any example herein, particularly any one of Examples 2-5, wherein the bore has a round shape.

Example 7: The medical implant of any example herein, particularly any one of Examples 1-6, wherein the radiopaque marker comprises opposite major surfaces, and wherein the edge surface extends between the opposite major surfaces.

Example 8: The medical implant according to any example herein, particularly Example 7, wherein the major surfaces are planar.

Example 9: The medical implant of any example herein, particularly any one of Examples 7-8, wherein the radiopaque marker is asymmetric about a plane transverse to the opposite major surfaces.

Example 10: The medical implant of any example herein, particularly any one of Examples 7-8, wherein the radiopaque marker is asymmetric about a plane transverse to the opposite major surfaces and intersecting both the recessed surface portion and the non-recessed surface portion.

Example 11: The medical implant of any example herein, particularly any one of Examples 1-10, wherein the frame comprises a plurality of struts including the at least one strut.

Example 12: The medical implant according to any example herein, particularly Example 11, wherein the medical implant comprises a docking station configured to receive a prosthetic valve.

Example 13: The medical implant according to any example herein, particularly Example 12, wherein the plurality of struts define a valve seat for engaging the prosthetic valve when the prosthetic valve is inserted into the docking station.

Example 14: The medical implant according to any example herein, particularly Example 13, further comprising a skirt attached to the valve seat.

Example 15: The medical implant according to any example herein, particularly Example 14, wherein the valve seat includes the at least one strut, and wherein the skirt covers the at least one strut.

Example 16: The medical implant of any example herein, particularly any one of Examples 14-15, further comprising a suture extending through the eyelet and the skirt.

Example 17: The medical implant of any example herein, particularly any one of Examples 1-16, wherein the medical implant is sterilized.

Example 18: A medical assembly comprising: a delivery apparatus comprising a handle and at least one shaft coupled to the handle; a frame coupled to the at least one shaft, the frame comprising at least one strut having a strut portion with a first concave surface and a second concave surface on opposite sides of the strut portion; and a radiopaque marker having an edge surface with a recessed surface portion and a non-recessed surface portion, the radiopaque marker coupled to the at least one strut with the recessed surface portion positioned in opposing relation to the first concave surface.

Example 19: The medical assembly according to any example herein, particularly Example 18, wherein the at least one strut includes a bore adjacent to the strut portion, wherein a portion of a wall of the bore forms the first concave surface, and wherein the radiopaque marker is disposed within the bore.

Example 20: The medical assembly according to any example herein, particularly any one of Examples 18-19, wherein the radiopaque marker engages the wall of the bore via the non-recessed surface portion.

Example 21: The medical assembly according to any example herein, particularly any one of Examples 18-20, wherein the at least one strut includes an eyelet adjacent to the strut portion, and wherein a portion of a wall of the eyelet forms the second concave surface.

Example 22: The medical assembly according to any example herein, particularly any one of Examples 18-21, wherein the bore has a round shape.

Example 23: The medical assembly according to any example herein, particularly any one of Examples 18-22, wherein the frame comprises a plurality of struts including the at least one strut, and wherein the plurality of struts define a valve seat for engaging a prosthetic valve.

Example 24: The medical assembly according to any example herein, particularly Example 23, further comprising a skirt attached to the valve seat, wherein the valve seat includes the at least one strut, and wherein the skirt covers the at least one strut.

Example 25: A docking station for a prosthetic valve, the docking station comprising: a frame comprising at least one strut in which a bore and an eyelet are formed, the at least one strut having a strut portion between the bore and the eyelet, wherein a portion of a wall of the bore forms a first surface of the strut portion and a portion of a wall of the eyelet forms a second surface of the strut portion; and a radiopaque marker disposed within the bore, the radiopaque marker having an edge surface with a recessed surface portion and a non-recessed surface portion, the recessed surface portion positioned in opposing relation to the first surface of the strut portion.

Example 26: The docking station according to any example herein, particularly Example 25, wherein the bore has a round shape.

Example 27: The docking station according to any example herein, particularly any one of Examples 25-26, wherein the recessed surface portion comprises an inwardly curved surface, and wherein the non-recessed surface portion comprises an outwardly curved surface.

Example 28: The docking station according to any example herein, particularly any one of Examples 25-27, wherein the radiopaque marker engages the wall of the bore only via the non-recessed surface portion.

Example 29: The docking station according to any example herein, particularly any one of Examples 25-28, wherein the bore and the eyelet are aligned along a longitudinal axis of the at least one strut, and wherein the radiopaque marker is asymmetric about the longitudinal axis.

Example 30: The docking station according to any example herein, particularly any one of Examples 25-29, further comprising a skirt attached to the frame, wherein the skirt encapsulates the at least one strut.

Example 31: A medical implant comprising: a frame having a plurality of struts defining a plurality of cells, at least one strut of the plurality of struts having a bore and an eyelet formed therein, the at least one strut having a strut portion between the bore and the eyelet, wherein a portion of a wall of the bore forms a first surface of the strut portion and a portion of the wall of the eyelet forms a second surface of the strut portion, wherein the first and second surfaces are opposite sides of the strut portion; and a radiopaque marker disposed within the bore, the radiopaque marker having an edge surface with a recessed surface portion and a non-recessed surface portion, the recessed surface portion positioned in opposing relation to the first surface of the strut portion.

Example 32: The medical implant according to any example herein, particularly Example 31, wherein the recessed surface portion comprises an inwardly curved surface, and wherein the non-recessed surface portion comprises an outwardly curved surface.

Example 33: The medical implant of any example herein, particularly any one of Examples 31-32, wherein the radiopaque marker engages the wall of the bore only via the non-recessed surface portion.

Example 34: The medical implant of any example herein, particularly any one of Examples 31-33, wherein the radiopaque marker comprises opposite major surfaces, and wherein the edge surface extends between the opposite major surfaces.

Example 35: The medical implant of any example herein, particularly any one of Examples 31-34, wherein the bore and the eyelet are aligned along a longitudinal axis of the at least one strut, and wherein the radiopaque marker is asymmetric about the longitudinal axis.

Example 36: The medical implant of any example herein, particularly any one of Examples 31-35, wherein the bore has a round shape.

Example 37: The medical implant of any example herein, particularly any one of Examples 31-36, further comprising a skirt attached to the frame with a suture extending through the eyelet.

Example 38: The medical implant of any example herein, particularly any one of Examples 31-37, wherein the medical implant comprises a docking station comprising a valve seat configured to receive a prosthetic valve.

Example 39: The medical implant according to any example herein, particularly Example 38, wherein the skirt is disposed around the valve seat.

Example 40: The medical implant according to any example herein, particularly Example 39, wherein the skirt covers the at least one strut and the radiopaque marker disposed within the bore of the at least one strut.

Example 41: A medical implant comprising: a frame having a first end, a second end, a longitudinal axis extending from the first end to the second end and defining an axial direction, and a plurality of bores formed in the frame at positions along a circumference of the frame; and a plurality of radiopaque markers disposed in the plurality of bores, a first radiopaque marker of the plurality of radiopaque markers having an edge surface defined by a recessed surface portion and a non-recessed surface portion, wherein the first radiopaque marker engages a portion of a wall of a first bore of the plurality of bores via the non-recessed surface portion.

Example 42: The medical implant according to any example herein, particularly Example 41, wherein a portion of the frame defines a valve seat, and wherein the plurality of bores are disposed circumferentially about the valve seat.

Example 43: The medical implant according to any example herein, particularly Example 42, wherein the portion of the frame comprises a plurality of axial struts spaced about the circumference of the frame, and wherein the plurality of bores are formed in at least some of the plurality of axial struts.

Example 44: The medical implant according to any example herein, particularly Example 43, further comprising a plurality of angled struts extending between and connected to the axial struts at strut junctions.

Example 45: The medical implant according to any example herein, particularly any one of Examples 43-44, further comprising a plurality of cantilever struts attached to the axial struts and defining an opening of the valve seat.

Example 46: The medical implant according to any example herein, particularly any one of Examples 41-45, further comprising a plurality of eyelets formed in the frame at positions along the circumference of the frame.

Example 47: The medical implant according to any example herein, particularly Example 46, wherein a first eyelet of the plurality of eyelets and a first bore of the plurality of bores are formed in a first axial strut of the plurality of axial struts.

Example 48: The medical implant according to any example herein, particularly Example 47, wherein the first axial strut includes a strut portion between the first bore and the first eyelet, wherein a portion of a wall of the first bore forms a first side of the strut portion and a portion of a wall of the first eyelet forms a second side of the strut portion, wherein the first and second sides are opposite sides of the strut portion

Example 49: The medical implant according to any example herein, particularly Example 48, wherein the recessed surface portion is positioned in opposing relation to the first side of the strut portion.

Example 50: The medical implant of any example herein, particularly any one of Examples 47-49, wherein the first bore and the first eyelet are aligned along a longitudinal axis of the first axial strut, and wherein the first radiopaque marker is asymmetric about the longitudinal axis.

Example 51: The medical implant according to any example herein, particularly any one of Examples 42-50, further comprising a skirt disposed around the valve seat.

Example 52: The medical implant according to any example herein, particularly Example 51, wherein the skirt covers the radiopaque markers.

Example 53: The medical implant of any example herein, particularly any one of Examples 41-52, wherein the first bore has a round shape.

Example 54: The medical implant of any example herein, particularly any one of Examples 41-53, wherein the recessed surface portion comprises an inwardly curved surface, and wherein the non-recessed surface portion comprises an outwardly curved surface.

Example 55: A medical assembly comprising: a delivery apparatus comprising a handle and at least one shaft coupled to the handle; a frame coupled to the at least one shaft, the frame having a first end, a second end, a longitudinal axis extending from the first end to the second end and defining an axial direction, and a plurality of bores formed in the frame at positions along a circumference of the frame; and a plurality of radiopaque markers disposed in the plurality of bores, a first radiopaque marker of the plurality of radiopaque markers having an edge surface defined by a recessed surface portion and a non-recessed surface portion, wherein the first radiopaque marker engages a portion of a wall of a first bore of the plurality of bores via the non-recessed surface portion.

Example 56: The medical assembly according to any example herein, particularly Example 55, wherein a portion of the frame defines a valve seat, and wherein the plurality of bores are disposed circumferentially about the valve seat.

Example 57: The medical assembly according to any example herein, particularly any one of Examples 55-56, further comprising a plurality of eyelets formed in the frame at positions along the circumference of the frame.

Example 58: The medical assembly according to any example herein, particularly Example 57, wherein the frame comprises a plurality of struts, and wherein the first bore and a first eyelet of the plurality of eyelets are formed in a first strut of the plurality of struts.

Example 59: The medical assembly according to any example herein, particularly Example 58, wherein the first strut includes a strut portion between the first bore and the first eyelet, wherein a portion of a wall of the first bore forms a first side of the strut portion and a portion of a wall of the first eyelet forms a second side of the strut portion, wherein the first and second sides are opposite sides of the strut portion

Example 60: The medical assembly according to any example herein, particularly Example 59, wherein the recessed surface portion is positioned in opposing relation to the first side of the strut portion.

Example 61: The medical assembly according to any example herein, particularly any one of Examples 58-60, wherein the first bore and the first eyelet are aligned along a longitudinal axis of the first strut, and wherein the first radiopaque marker is asymmetric about the longitudinal axis.

Example 62: The medical assembly according to any example herein, particularly any one of Examples 56-61, further comprising a skirt disposed around the valve seat.

Example 63: The medical assembly according to any example herein, particularly Example 62, wherein the skirt covers the radiopaque markers.

Example 64: The medical assembly according to any example herein, particularly any one of Examples 55-63, wherein the first bore has a round shape.

Example 65: The medical assembly according to any example herein, particularly any one of Examples 55-64, wherein the recessed surface portion comprises an inwardly curved surface, and wherein the non-recessed surface portion comprises an outwardly curved surface.

Example 66: A medical implant comprising: a frame comprising at least one strut in which a bore and an eyelet are formed, the at least one strut having a strut portion between the bore and the eyelet, wherein a portion of a wall of the bore forms a first surface of the strut portion and a portion of a wall of the eyelet forms a second surface of the strut portion; and a radiopaque marker disposed within the bore, the radiopaque marker having a wavy edge surface defined by a series of recessed surface portions and a series of non-recessed surface portions, wherein the series of non-recessed surface portions engage the wall of the bore.

Example 67: The medical implant according to any example herein, particularly Example 66, wherein the bore has a round shape.

Example 68: The medical implant according to any example herein, particularly Example 66, wherein the wall of the bore has a wavy profile complementary to the wavy edge surface.

Example 69: The medical implant according to any example herein, particularly Example 68, wherein the series of recessed surface portions and the series of non-recessed surface portions engage the wall of the bore.

Example 70: The medical implant of any example herein, particularly any one of Examples 66-69, wherein the frame comprises a plurality of struts including the at least one strut.

Example 71: The medical implant according to any example herein, particularly Example 70, wherein the medical implant comprises a docking station configured to receive a prosthetic valve.

Example 72: The medical implant according to any example herein, particularly Example 71, wherein the plurality of struts define a valve seat for engaging the prosthetic valve when the prosthetic valve is inserted into the docking station.

Example 73: The medical implant according to any example herein, particularly Example 72, further comprising a skirt attached to the valve seat.

Example 74: The medical implant according to any example herein, particularly Example 73, wherein the valve seat includes the at least one strut, and wherein the skirt covers the at least one strut.

Example 75: The medical implant of any example herein, particularly any one of Examples 66-74, further comprising a suture extending through the eyelet and the skirt.

Example 76: A method of assembling a medical device, the method comprising: attaching a radiopaque marker to a strut of a frame having a strut portion with opposite first and second concave surfaces, wherein attaching the radiopaque marker comprises positioning a recessed surface portion of an edge surface of the radiopaque marker in opposing relation to one of the first and second concave surfaces.

Example 77: The method according to any example herein, particularly Example 76, further comprising attaching a skirt to the frame, wherein the skirt covers the strut.

Example 78: The method according to any example herein, particularly any one of Examples 76-77, wherein the attaching the radiopaque marker further comprises disposing the radiopaque marker within a bore in the strut, wherein a portion of a wall of the bore forms the one of the first and second concave surfaces.

Example 79: The method according to any example herein, particularly Example 78, further comprising attaching a non-recessed surface portion of the edge surface of the radiopaque marker to the wall of the bore.

Example 80: A method comprising: coupling a docking station to a distal end of a delivery apparatus, the docking station comprising a plurality of radiopaque markers disposed in a plurality of bores, wherein at least one of the radiopaque markers has an edge surface defined by a recessed surface portion and a non-recessed surface portion, wherein the at least one of the radiopaque markers engages a portion of a wall of one of the plurality of bores on only the non-recessed surface portion; inserting the distal end of the delivery apparatus into a patient's vasculature; advancing the delivery apparatus through the patient's vasculature to position the docking station at an implantation site; positioning the docking station at the implantation site with the aid of the radiopaque markers; and expanding the docking station.

Example 81: The method according to any example herein, particularly Example 80, further comprising delivering a prosthetic valve to the implantation site and positioning the prosthetic valve in a valve seat of the docking station with the aid of the radiopaque makers.

Example 82: A medical implant comprises a frame having a plurality of struts, at least one strut of the plurality of struts having a bore formed therein; and a non-circular radiopaque marker fitted within the bore and having an edge surface frictionally engaged with a wall of the bore, wherein the edge surface has a non-cylindrical surface profile configured to frictionally engage the wall of the bore in a manner to reduce strain concentration on the wall of the bore due to an outward radial pressure exerted on the wall of the bore by the radiopaque marker.

Example 83: The medical implant according to any example herein, particularly Example 82, wherein the edge surface includes a recessed surface portion and a non-recessed surface portion defining the non-cylindrical surface profile, and wherein the edge surface frictionally engages the wall only via the non-recessed surface portion.

Example 84: The medical implant according to any example herein, particularly Example 83, wherein the at least one strut includes an eyelet and a strut portion having a first surface forming a part of the wall of the bore and a second surface forming a part of a wall of the eyelet, and wherein the radiopaque marker is oriented within the bore such that the recessed surface portion is positioned in opposing relation to the first surface of the strut portion.

Example 85: The medical implant according to any example herein, particularly Example 82, wherein the edge surface comprises a series of recessed surface portions and a series of non-recessed surface portions defining the non-cylindrical surface profile.

Example 86: The medical implant according to any example herein, particularly Example 85, wherein the wall of the bore has a non-cylindrical surface profile that is complementary to the non-cylindrical surface profile of the edge surface.

Example 87: The medical implant according to any example herein, particularly any one of Examples 82-85, wherein the bore has a round shape.

Example 88: A medical assembly comprising: a delivery apparatus comprising a handle and at least one shaft coupled to the handle; a frame coupled to the at least one shaft, the frame having a plurality of struts, at least one strut of the plurality of struts having a bore formed therein; and a non-circular radiopaque marker fitted within the bore and having an edge surface frictionally engaged with a wall of the bore, wherein the edge surface has a non-cylindrical surface profile configured to frictionally engage the wall of the bore in a manner to reduce strain concentration on the wall of the bore due to an outward radial pressure exerted on the wall of the bore by the radiopaque marker.

Example 89: A method comprising: coupling a docking station to a distal end of a delivery apparatus, the docking station comprising a plurality of radiopaque markers disposed in a plurality of bores, wherein at least one of the radiopaque markers has an edge surface with a non-cylindrical surface profile configured to engage a wall of one of the bores in a manner to reduce strain concentration around the one of the bores due to outward radial pressure exerted on the one of the bores by the at least one of the radiopaque markers; inserting the distal end of the delivery apparatus into a patient's vasculature; advancing the delivery apparatus through the patient's vasculature to position the docking station at an implantation site; positioning the docking station at the implantation site with the aid of the radiopaque markers; and expanding the docking station.

Example 90: The method according to any example herein, particularly Example 89, further comprising delivering a prosthetic valve to the implantation site and positioning the prosthetic valve in a valve seat of the docking station with the aid of the radiopaque makers.

Example 91: A medical implant comprising: a metal frame having a plurality of struts, at least one strut of the plurality of struts having a bore formed therein; and a radiopaque marker fitted within the bore; wherein one of the bore and the radiopaque marker has a cylindrical surface profile and the other of the bore and the radiopaque marker has a non-cylindrical surface profile, or both the bore and the radiopaque marker have non-cylindrical surface profiles.

Example 92: The medical implant according to any example herein, particularly Example 91, wherein a wall of the bore has a non-cylindrical surface profile comprising a plurality of radial projections spaced about a circumference of the bore, and wherein an edge surface of the radiopaque marker frictionally engages the radial projections on the wall.

Example 93: The medical implant according to any example herein, particularly Example 92, wherein the edge surface has a cylindrical profile.

Example 94: The medical implant according to any example herein, particularly Example 92, wherein the edge surface has a non-cylindrical surface profile comprising a plurality of radial projections that extend into recesses between the plurality of radial projections of the wall.

Example 95: The medical implant according to any example herein, particularly Example 91, wherein an edge surface of the radiopaque marker has a non-cylindrical surface profile comprising a recessed surface portion and a non-recessed surface portion.

Example 96: The medical implant according to any example herein, particularly any one of Examples 91-95, wherein the strut comprises an eyelet adjacent the bore.

Example 97: The medical implant according to any example herein, particularly Example 96, wherein a suture extends through the eyelet.

Example 98: A method of assembling a medical device comprising: fixing a radiopaque marker within a bore of a strut of a metal frame; wherein one of the bore and the radiopaque marker has a cylindrical surface profile and the other of the bore and the radiopaque marker has a non-cylindrical surface profile, or both the bore and the marker have non-cylindrical surface profiles.

Example 99: The method of assembling a medical device according to any example herein, particularly Example 98, wherein fixing the radiopaque marker within the bore comprises compressing opposing surfaces of the radiopaque marker to reduce a height of the radiopaque marker and expand the radiopaque marker radially such that the radiopaque marker frictionally engages the bore.

Example 100: A method comprising: sterilizing the medical implant of any example herein, particularly any one of Examples 1-17, Examples 31-54, Examples 66-75, Examples 82-87, and Examples 91-97.

Example 101: The medical implant of any example herein, particularly any one of Examples 31-54, Examples 66-75, Examples 82-87, and Examples 91-97, wherein the medical implant is sterilized

Example 102: A method comprising: sterilizing the medical assembly of any example herein, particularly any one of Examples 18-24, Examples 55-65, and Example 88.

Example 103: The medical assembly of any example herein, particularly any one of Examples 18-24, Examples 55-65, and Example 88, wherein the medical assembly is sterilized.

Example 104: A method comprising: sterilizing the docking station of any example herein, particularly any one of Examples 25-30.

Example 105: The docking station of any example herein, particularly any one of Examples 25-30, wherein the docking station is sterilized.

The features described herein with regard to any example can be combined with other features described in any one or more of the other examples, unless otherwise stated. For example, any one or more of the features of one medical implant can be combined with any one or more features of another medical implant. As another example, any one or more features of one medical assembly can be combined with any one or more features of another medical assembly.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A medical implant comprising:
a frame comprising at least one strut having a strut portion with a first concave surface and a second concave surface on opposite sides of the strut portion; and
a radiopaque marker having an edge surface with a recessed surface portion and a non-recessed surface portion, the radiopaque marker coupled to the at least one strut with the recessed surface portion positioned in opposing relation to the first concave surface.

2. The medical implant of claim 1, wherein the at least one strut includes a bore adjacent to the strut portion, wherein a portion of a wall of the bore forms the first concave surface, and wherein the radiopaque marker is disposed within the bore.

3. The medical implant of claim 2, wherein the non-recessed surface portion engages the wall of the bore.

4. The medical implant of claim 2, wherein a longitudinal axis of the strut extends through the first and second concave surfaces, and wherein the radiopaque marker is asymmetric about the longitudinal axis of the strut.

5. The medical implant of claim 2, wherein the at least one strut includes an eyelet adjacent to the strut portion, and wherein a portion of a wall of the eyelet forms the second concave surface.

6. The medical implant of claim 1, wherein the radiopaque marker comprises opposite planar major surfaces, and wherein the edge surface extends between the opposite planar major surfaces.

7. The medical implant of claim 6, wherein the radiopaque marker is asymmetric about a plane transverse to the opposite planar major surfaces.

8. The medical implant of claim 1, wherein the frame comprises a plurality of struts including the at least one strut, wherein the medical implant comprises a docking station configured to receive a prosthetic valve, and wherein the plurality of struts define a valve seat for engaging the prosthetic valve when the prosthetic valve is inserted into the docking station.

9. The medical implant of claim 8, further comprising a skirt attached to the valve seat.

10. A medical assembly comprising:
a delivery apparatus comprising a handle and at least one shaft coupled to the handle;
a frame coupled to the at least one shaft, the frame comprising at least one strut having a strut portion with a first concave surface and a second concave surface on opposite sides of the strut portion; and
a radiopaque marker having an edge surface with a recessed surface portion and a non-recessed surface portion, the radiopaque marker coupled to the at least one strut with the recessed surface portion positioned in opposing relation to the first concave surface.

11. The medical assembly of claim 10, wherein the at least one strut includes a bore adjacent to the strut portion, wherein a portion of a wall of the bore forms the first concave surface, and wherein the radiopaque marker is disposed within the bore.

12. The medical assembly of claim 11, wherein the radiopaque marker engages the wall of the bore via the non-recessed surface portion.

13. The medical assembly of claim 11, wherein the bore has a round shape.

14. The medical assembly of claim 10, wherein the frame comprises a plurality of struts including the at least one strut, and wherein the plurality of struts define a valve seat for engaging a prosthetic valve.

15. A medical implant comprising:
a frame having a plurality of struts defining a plurality of cells, at least one strut of the plurality of struts having a bore and an eyelet formed therein, the at least one strut having a strut portion between the bore and the eyelet, wherein a portion of a wall of the bore forms a first surface of the strut portion and a portion of the wall of the eyelet forms a second surface of the strut portion, wherein the first and second surfaces are opposite sides of the strut portion; and
a radiopaque marker disposed within the bore, the radiopaque marker having an edge surface with a recessed surface portion and a non-recessed surface portion, the recessed surface portion positioned in opposing relation to the first surface of the strut portion.

16. The medical implant of claim 15, wherein the recessed surface portion comprises an inwardly curved surface, and wherein the non-recessed surface portion comprises an outwardly curved surface.

17. The medical implant of claim 15, wherein the radiopaque marker engages the wall of the bore only via the non-recessed surface portion.

18. The medical implant of claim 15, further comprising a skirt attached to the frame with a suture extending through the eyelet.

19. The medical implant of claim 15, wherein the medical implant comprises a docking station comprising a valve seat configured to receive a prosthetic valve, and wherein the skirt is disposed around the valve seat.

20. The medical implant of claim 19, wherein the skirt covers the at least one strut and the radiopaque marker disposed within the bore of the at least one strut.
